(19) United States
(12) Patent Application Publication
(10) Pub. No.: US 2006/0121045 A1
(43) Pub. Date: Jun. 8, 2006

(54) Antibody fragments for protection from pathogen infection and methods of use thereof

(76) Inventors: Brent Iverson, Austin, TX (US); George Georgiou, Austin, TX (US); Robert Mabry, Austin, TX (US)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 11/270,894

(22) Filed: Nov. 8, 2005

Related U.S. Application Data
(60) Provisional application No. 60/626,141, filed on Nov. 8, 2004.

(51) Int. Cl. A61K 39/40; A61K 39/07

(52) U.S. Cl. 424/164.1; 424/246.1

(57) ABSTRACT

Methods and compositions are provided for an antibody fragment or functional fragment thereof, which fragments do not contain an Fc region. The invention also describes in vitro and in vivo treatment of a subject exposed to a pathogen, pathogen infection, or pathogen protein product by administering to the subject a therapeutically effective amount of the described antibody fragments and functional fragments thereof.

### CROSS REFERENCE TO RELATED APPLICATION(S)

This application claims the benefit of priority under 35 U.S.C. § 119(e) of U.S. Ser. No. 60/626,141, filed Nov. 8, 2004, which is incorporated herein by reference in its entirety.

### GRANT INFORMATION

This invention was made in part with government support under an NIH grant, U01 AI056431. The United States government has certain rights in this invention.

### FIELD OF THE INVENTION

The present invention relates generally to antibody fragments or functional fragments thereof useful and more specifically, to antibody fragments or functional fragments thereof that bind to anthrax protective antigen, and methods of producing such fragments in a bacterial expression system.

### BACKGROUND INFORMATION

Research on the spore forming bacterium Bacillus anthracis is limited due to its rare occurrence in humans, because most anthrax infections occur in hoofed mammals. In humans, the preventive treatment strategy is generally limited to the use of a few antibiotics, including penicillin, doxycycline and fluroquinolones. While an anthrax vaccine can prevent infection, the Advisory Committee on Immunization Practices (Centers for Disease Control and Prevention (CDC)) recommends anthrax vaccination for selected groups of society, for example: 1) Persons who work directly with the organism in the laboratory; 2) Persons who work with imported animal hides or furs in areas where standards are insufficient to prevent exposure to anthrax spores; 3) Persons who handle potentially infected animal products in high-incidence areas; and 3) Military personnel deployed to areas with high risk for exposure to the organism (as when it is used as a biological warfare weapon). Hence, the CDC does not recommend widespread immunization for the general public. In fact, vaccination for the general public is not available.

### BRIEF DESCRIPTION OF THE DRAWINGS

FIG. 1 is a sequence alignment showing the anti-PA 14B7 scFv amino acid sequence.

FIG. 2 is a graph showing in vitro toxin challenge.

FIG. 3 is a graph showing pharmacokinetic analysis of Hartley guinea pigs injected subcutaneously with 14B7 IgG, M18 scAb conjugated to PEG 20 kDa, or M18 scAb conjugated to PEG 40 kDa.

FIG. 4 is a graph showing in vivo inhalation anthrax spore challenge.

### SUMMARY OF THE INVENTION

The present invention is based on the seminal discovery that antibody fragments are useful for protection against infection, even in the absence of an Fc region. The invention methods and compositions described herein solve the dilemma of how to produce a robust amount of a therapeutic antibody fragments or functional fragments thereof. This is the first example of the therapeutic effects of an antibody fragments or functional fragments thereof, excluding the Fc region.

In one embodiment of the invention, there is provided a method of protecting a subject against challenge from a pathogen or pathogen protein product thereof by administering to the subject a therapeutically effective amount of an antibody fragment or functional fragment thereof, with the proviso that the fragment does not contain an Fc region. Further, the antibody fragments or functional fragments thereof described herein can be optionally conjugated to a non-protein polymer, e.g., polyethylene-glycol (PEG).

In another embodiment, the invention provides methods of producing an antibody fragments or functional fragments thereof that is protective when administered to a subject at risk of being infected by a pathogen or pathogen protein product thereof, comprising: a) introducing an expression vector containing a nucleic acid encoding the antibody fragment protein or functional fragment thereof, into the bacterial cell; and b) culturing the bacterial cell under conditions whereby the antibody fragment protein or functional fragment thereof is expressed, thereby producing the antibody fragments or functional fragments thereof that is protective when administered to a subject at risk of being infected by a pathogen or protein product thereof.

In another embodiment, the invention provides methods of producing antibody fragments or functional fragments thereof as described above, by culturing a recombinant bacterial cell containing a nucleic acid encoding an antibody fragment or functional fragment thereof that binds to anthrax protective antigen (PA) toxin protein, such that the protein is expressed by the bacterial cell.

In another embodiment, a purified antibody fragment or functional fragment thereof that specifically binds to anthrax protective antigen (PA) toxin is provided, wherein the antibody fragment or functional fragments thereof is as set forth in SEQ ID NO: 1, and further including mutations 121V, L46F, S56P, S76N, Q78L, and L94P of the light chain, and S30N, T57S, K64E, and T681 of the heavy chain; Q38R, Q55L, S56P, T74A and Q78L of the light chain, and K62R of the heavy chain; and S22G, L33 S, Q55L, S56P, Q78L and L94P of the light chain, and S7P, K19R, S30N, T681, and M80L of the heavy chain, wherein the numbering of the amino acid is based on SEQ ID NO: 1.

### DETAILED DESCRIPTION OF THE INVENTION

The present invention is based on the seminal discovery that antibody fragments or functional fragments thereof, lacking the Fc region, provide a therapeutic effect when administered to a subject. The antibody fragments or functional fragments thereof of the present invention are efficiently expressed in bacterial systems, and because they do not contain the Fc regions as compared to whole antibodies, they do not activate the complement system. In an illustrative example, the invention provides antibody fragments or functional fragments thereof that bind to anthrax PA toxin and protect against subsequent challenge in vitro and in vivo challenge with Bacillus anthracis spores.

Bacillus anthracis is a spore-forming, gram-positive bacterium that causes anthrax. Upon entry through the skin, ingestion, or inhalation, B. anthracis spores germinate into vegetative bacteria. A tripartite exotoxin secreted from the bacteria represents a key virulence factor in anthrax. The anthrax PA component of the exotoxin mediates the host cell entry of the two other components, the lethal factor (LF), a zinc metalloprotease that cleaves several mitogen-activated protein kinase kinases, and the edema factor, a calmodulin-dependent adenylate cyclase. Structures of all three proteins have been determined. In addition, the mechanisms by which the PA-LF complex (lethal toxin [LeTx]) enters the cell have been identified along with the chronology with which these events occur. The protective antigen binds to two cell surface receptors, ATR and CMG2, and suggests that the CMG2 gene is expressed in most human tissues and, recently, the ATR/TEM8 gene was reported to be highly expressed in epithelial cells.

For persons infected with anthrax, treatment success is limited by several factors, such as the increased incidence of antibiotic resistance and treatment delays that lessen the chance of survival. It is known that early treatment of anthrax with antibiotics is essential to reduce mortality-delays in treatment profoundly decrease survival rates. Early treatment, however, is difficult because initial symptoms of the infection, e.g., when the bacterial spores are inhaled, heretofore known as inhalation anthrax, may resemble those of the common cold. In addition, symptoms of anthrax infection, depending on how the bacterium is contracted, may take seven to sixty days to appear.

The invention described herein, provides a method of treating subjects with the pathogen or are risk of exposure to the pathogen, by neutralization of PA by long-circulating, ultra-high-affinity antibody fragments, and thereby conferring protection against the pathogen (e.g., anthrax) despite the absence of Fc-mediated immune responses. Benefits of such a strategy include, but are not limited to: (i) substantially lower costs of manufacturing antibody fragments. This is a critical issue since the current prophylactic IgG antibodies require large dosages (4 to 10 mg/kg of body weight) and must be produced by recombinant CHO cells, (ii) elimination of potentially adverse effects associated with Fc, and (iii) rapid production in bacterial cells.

The present invention relates to methods of protecting or treating or ameliorating a subject against pathogenic challenge or having a pathogenic infection by administering to the subject a therapeutically effective amount of the antibody fragments or functional fragments thereof as described herein.

As used herein, the term “treatment” means any manner in which the symptoms of a condition, disorder or disease are ameliorated or otherwise beneficially altered. Treatment also encompasses any pharmaceutical use of the compositions herein, such as use as antibacterial agents, agonists and/or antagonists. Also, as used herein, the term “amelioration” of the symptoms of a particular disorder by administration of a particular pharmaceutical composition refers to any lessening, whether permanent or temporary, lasting or transient that can be attributed to or associated with administration of the composition.

The treatment and amelioration provided by the invention is accomplished by administering a therapeutically effective amount of the antibody composition described herein. As used herein, the term “a therapeutically effective amount” for treating a particular disease, for example, a pathogen or pathogenic infection, means an amount that is sufficient to ameliorate, or in some manner reduce the symptoms associated with the disease. Such amount may be administered as a single dosage or may be administered according to a regimen, whereby it is effective. The amount may cure the disease and/or ameliorate the symptoms of the disease. Repeated administration may be required to achieve the desired amelioration of symptoms.

The antibody fragments or functional fragments thereof described herein are used to treat a pathogen, pathogenic infection, or pathogen protein product. The pathogen or pathogen protein product, from which epitopes are derived, can be any protein product for which it is desired to identify an immunogenic peptide. In one aspect, the protein product is a protein of an infectious microorganism, for example, a cell surface protein, a toxin, a protein involved in infectiousness or spread of the microorganism, or any other protein of the microorganism, particularly a protein that, in a living subject or a sample obtained from a living subject, can be contacted with an antibody.

The compositions and methods of the present invention have application to a wide variety of pathogens including, but not limited to, eukaryotic or prokaryotic infectious microorganism, including, for example, a bacterium, a protozoan, a yeast, or a fungus. Bacterial infections such as native bacterial strains that produce anthrax, diptheria, pertussis, tetanus, and E. coli strains producing Shiga toxin. For example, the infectious microorganism can be a bacterium that causes anthrax (e.g., Bacillus anthracis), in which case the protein product can be an anthrax protective antigen (PA) or an anthrax lethal factor or both. Other pathogenic bacteria include Brucella aborts, Brucella melitensis, Brucella suds, Chlamydia psittaci, Clostridium botulinum, Francisella tularensis, Pseudomonas mallet, Pseudomonas pseudomallei, Salmonella typhi, Shigella dysenteriae and Vibrio cholera.

In one aspect of the invention, representative examples of antigens which can be used to produce an immune response using the methods of the present invention include various pathogens, for example, influenza hemagglutinin, influenza nuclear protein, influenza M2, tetanus toxin C-fragment, anthrax protective antigen, anthrax lethal factor, rabies glycoprotein, HBV surface antigen, HIV gp 120, HIV gp 160, human carcinoembryonic antigen, malaria CSP, malaria SSP, malaria MSP, malaria pfg, and mycobacterium tuberculosis HSP, and the like. In one aspect, the immune response produces a protective effect against infectious pathogens, or pathogen protein product(s), for example, the anthrax protective antigen (PA). The anthrax PA antigen has been reported, for example, see Welkos, S. L. et al. (1988) “Sequence and analysis of the DNA encoding protective antigen of Bacillus anthracis,” Gene 69(2):287-300; Price, L. B. et al. (1999) “Genetic diversity in the protective antigen gene of Bacillus anthracis,” J. Bacteriol. 181 (8), 2358-2362; and Maynard et al. (2002), “Protection against anthrax toxin by recombinant antibody fragments correlates with antigen affinity,” Nature Biotechnology 20:597-601, which are incorporated herein by reference in their entirety. Thus, the invention also provides methods wherein the nucleic acid molecule can encode an epitope of interest and/or an antigen of interest and/or a nucleic acid molecule that stimulates and/or modulates an immunological response and/or stimulates and/or modulates expression, e.g., transcription and/or translation, such as transcription and/or translation of an endogenous and/or exogenous nucleic acid molecule; and/or elicits a therapeutic response.

The anthrax PA is the dominant antigen in both natural and vaccine-induced immunity to anthrax infection. It is also essential for host cell intoxication in combination with either lethal factor (LF) or edema factor (EF), producing lethal toxin or edema toxin, respectively. The anthrax “protective antigen” (PA) is an 83 kDa protein produced by Bacillus anthracis. PA is one of two protein components of the lethal or anthrax toxin produced by B. anthracis. The 83 kDa PA binds at its carboxyl-terminus to a cell surface receptor, where it is specifically cleaved by a protease, e.g., furin, clostripain, or trypsin. This enzymatic cleavage releases a 20 kDa amino-terminal PA fragment, while a 63 kDa carboxyl-terminal PA fragment remains bound to the cell surface receptor. The description of protective antigen includes binary toxin functional equivalents and other epitopes of interest, e.g., the a 63 kDa PA fragment that results from the enzymatic cleavage of the 83 kDa PA. Processed PA contains both a cell surface receptor binding site at its carboxyl-terminus and a lethal factor binding site at its new amino-terminus. Processed PA may be produced by enzymatic cleavage in vitro or in vivo, or as a recombinant protein. The description of processed PA also includes binary toxin functional equivalents. The antigens described herein also include the anthrax lethal factor (LF), a 90 kDa protein that is the second protein component, along with PA, of the B. anthracis lethal or anthrax toxin. LF contains a PA binding site. The description of LF includes binary toxin functional equivalents.

For example, the protein product of the invention consists of a polypeptide which binds to an antibody fragment as set forth in, and/or optionally a polypeptide which binds to an antibody fragment as set forth in SEQ ID NO: 1 further having various mutations, including, but not limited to 121V, L46F, S56P, S76N, Q78L, and L94P of the light chain, and S30N, T57S, K64E, and T681 of the heavy chain; Q38R, Q55L, S56P, T74A and Q78L of the light chain, and K62R; and S22G, L33S, Q55L, S56P, Q78L and L94P of the light chain, and S7P, K19R, S30N, T681, and M80L of the heavy chain. The amino acid positions are based on the numbering system as set forth in SEQ ID NO: 1.

The invention herein relates not only to antibody polypeptides, peptides and peptide derivatives, but also to functional fragments thereof. As used herein, “antibody fragment” includes any fragments including an antibody fragment which consists essentially of pooled fragments made from monoclonal antibody production and with different epitopic specificities are provided. Also, as used herein, “functional fragments” means those antibody polypeptides not containing an Fc region which are capable of protecting, treating, ameliorating, or reducing the affect of a pathogen, pathogenic infection or pathogen protein product. Hence, an antibody functional fragment as set forth in SEQ ID NO: 1 may include any combination of the various mutations described herein, so long as the antibody fragment is capable of protecting, treating, ameliorating, or reducing the affect of a pathogen, pathogenic infection or pathogen protein product.

For example, the antibody polypeptide and functional fragments thereof may have changes in the amino acid sequence of the protein product are contemplated in the present invention. In one aspect of the invention, antibody fragment polypeptides and functional fragments thereof can be altered by changing the DNA encoding the polypeptides or functional fragments thereof. Additionally, antibody fragments or functional fragments thereof can have conservative amino acid mutations or alterations can also be undertaken using amino acids that have the same or similar properties. Illustrative amino acid substitutions include the changes of alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine. Still, other variants useful for the present invention comprise analogs, homologs, muteins and mimetics of the antibody fragment that retain the ability to specifically bind to their respective epitope, pathogen, pathogen protein product, or antigen.

In another aspect of the invention, the antibody fragment directed to the pathogen or pathogen protein product is an agonist or antagonist. As used herein, the terms “agonists” and “antagonists” are molecules that modulate signal transduction via a receptor or ion channel. Hence, the agonists and antagonists describe herein may optionally reduce, decrease or neutralize an immune response by binding to a cell surface receptor, though not necessarily at the binding site of the natural ligand, and can modulate signal transduction when used alone (i.e. can be surrogate ligands, or can alter signal transduction in the presence of the natural ligand, either to enhance or inhibit signaling by the natural ligand). Another class of antagonists may not bind directly to the receptor. Rather, they act on one or more downstream target molecules of the activated receptor or ion channel, thereby modulating signal transduction of the receptor or ion channel. For example, “antagonists” can be molecules that block or decrease the signal transduction activity of receptor, e.g., they can competitively, noncompetitively, and/or allosterically inhibit signal transduction from the receptor, whereas “agonists” potentiate, induce or otherwise enhance the signal transduction activity of a receptor.

Also, the antibody fragments or functional fragments thereof described herein may optionally contain amino acid substitutions, or linkers, including those substitutions which enable the site-specific coupling of at least one non-protein polymer, such as polypropylene glycol, polyoxyalkylene, or PEG molecule. It should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including MPEG, bifunctional PEG, multiarmed PEG, forked PEG, branched PEG, pendent PEG (i.e. PEG or related polymers having one or more functional groups pendent to the polymer backbone), or PEG with degradable linkages therein.

PEG is typically clear, colorless, odorless, soluble in water, stable to heat, inert to many chemical agents, does not hydrolyze or deteriorate, and is generally non-toxic. Poly(ethylene glycol) is considered to be biocompatible, which is to say that PEG is capable of coexistence with living tissues or organisms without causing harm. More specifically, PEG is substantially non-immunogenic, which is to say that PEG does not tend to produce an immune response in the body. When attached to a molecule having some desirable function in the body, such as a biologically active agent, the PEG tends to mask the agent and can reduce or eliminate any immune response so that an organism can tolerate the presence of the agent. PEG conjugates tend not to produce a substantial immune response or cause clotting or other undesirable effects. PEG having the formula —CH₂CH₂O—(CH₂CH₂O)ₙ—CH₂CH₂—, where n is from about 2 to about 4000, typically from about 20 to about 2000, is one useful polymer in the practice of the invention. PEG having a molecular weight of from about 800 Da to about 100,000 Da are particularly useful as the polymer backbone.

In another aspect of the invention, the antibody fragments or functional fragments thereof are conjugated to a non-protein polymer (e.g., PEG). As used herein, the term “conjugate” is intended to refer to the covalent attachment of a molecule, such as a biologically active molecule, to a polymer molecule, preferably poly(ethylene glycol). Covalent attachment means either direct coupling of the polymer to the molecule or coupling through a linker or spacer moiety.

PEGylation increases or prolongs antibody circulation in the subject, e.g., in the animal, the mammal, the human and the like. PEGylation is performed substantially as described in Pannifer et al. (2001). See Pannifer et al. (2001) Nature 414:229-233. However, other methods of conjugation or attaching a non-protein polymer are encompassed by the invention. Non-protein polymers such as PEG are used as a conjugating agent for enhancing pharmacokinetics/bioavailability, improving stability issues, and decreasing immunogenicity among other attributes for therapeutics have been described and are encompassed by the invention. For example, PEGylation of Fab fragments has provided increased serum half-lives and efficacy for antibody therapy for a variety of applications. The availability of maleimide-PEG has allowed conjugation to antibodies through reaction with free native or engineered cysteines as described herein. This chemistry has proven to be suitable not only for Fab but also for single-chain variable fragments (scFvs), immunoliposomes, and other conjugates.

As used herein, the term “PEGylate” means to attach at least one PEG molecule to a moiety, for example, an amino acid moiety. The invention encompasses both non-site and site-specific coupling or conjugation of PEG The terms “moiety”, “active moiety”, “activating group”, “reactive site”, “chemically reactive group”, “chemically reactive moiety” and “functional group” are used in the art and herein to refer to distinct, definable portions or units of a molecule, for example, a polypeptide molecule as described herein. The terms are somewhat synonymous in the chemical arts and are used herein to indicate the portions of molecules that perform some function or activity and are reactive with other molecules.

Further, PEG and related polymers may include degradable linkages in the polymer backbone or in the linker group between the polymer backbone and one or more of the terminal functional groups of the polymer molecule. The term “linkage” or “linker” is used herein to refer to groups or bonds that normally are formed as the result of a chemical reaction and typically are covalent linkages. Hydrolytically stable linkages means that the linkages are substantially stable in water and do not react with water at useful pHs, e.g., under physiological conditions for an extended period of time, perhaps even indefinitely. Hydrolytically unstable or degradable linkages means that the linkages are degradable in water or in aqueous solutions, including for example, blood. Enzymatically unstable or degradable linkages means that the linkage can be degraded by one or more enzymes.

The linker molecule can be a small bifunctional molecule, which can rapidly react with the OH group on a serine residue of an antibody fragment. This linker molecule is preferably a heterobifunctional linker molecule, such as an amino acid, which forms an ester with a serine residue of an antibody fragment. The second functional group of the linker molecule serve as the site for PEGylation by the PEGylating agent. The amino acid, glycine, is a preferred heterobifunctional linker molecule according to the present invention. Other suitable linker molecules can be readily recognized or determined by those of skill in the art.

For example, ester linkages formed by the reaction of PEG carboxylic acids or activated PEG carboxylic acids with alcohol groups on a biologically active agent generally hydrolyze under physiological conditions to release the agent. Other hydrolytically degradable linkages include carbonate linkages; imine linkages resulted from reaction of an amine and an aldehyde (see, e.g., Ouchi et al., Polymer Preprints, 38(1):582-3 (1997), which is incorporated herein by reference); phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, e.g., at an end of a polymer such as PEG, and a carboxyl group of a peptide; and oligonucleotide linkages formed by a phosphoramidite group, e.g., at the end of a polymer, and a 5′ hydroxyl group of an oligonucleotide. Accordingly, a linker is conjugated to both the non-protein polymer and to the polypeptide molecule, or amino residue of the polypeptide molecule, e.g., cysteine residue of an antibody fragment.

The “PEGylating agent” as used in the present application means any PEG derivative, which is capable of reacting with the OH of a serine residue or a functional group of a bifunctional linker molecule, such as the amino group of an amino acid linker molecule. The other functional group of the linker molecule serves to form a covalent linkage to the serine residue of the antibody fragment described herein, i.e., the carboxyl group of an amino acid linker molecule forms an ester linkage with serine. It can be an alkylating reagent, such as PEG aldehyde, PEG epoxide or PEG tresylate, or it can be an acylating reagent, such as PEG-O—(CH₂)ₙ CO₂-Z where n=1-3 and Z is N-succinimidyl or other suitable activating group. Also, it has been reported that higher molecular weight PEG are better for obtaining therapeutic efficacy in certain cases.

The PEGylating agent can be used in its mono-methoxylated form where only one terminus is available for conjugation, or in a bifunctional form where both termini are available for conjugation, such as for example in forming a conjugate with two antibody fragments or functional fragments thereof covalently attached to a single PEG moiety. If the PEGylating agent is an acylating agent, it can contain either a norleucine or ornithine residue bound to the PEG unit via an amide linkage. These residues allow a precise determination of the linked PEG units per mole of peptide (see for example Sartore et al., 1991). A solvent for the PEGylation reaction is preferably a polar aprotic solvent, such as DMF, DMSO, pyridine, and the like.

Methods providing for efficient PEGylation are well known in the art. See Francis et al., In: Stability of protein pharmaceuticals: in vivo pathways of degradation and strategies for protein stabilization (Eds. Ahern., T. and Manning, M. C.) Plenum, N.Y., 1991). Also, Delgado et al., “Coupling of PEG to Protein By Activation With Tresyl Chloride, Applications In Immunoaffinity Cell Preparation”, In: Fisher et al., eds., Separations Using Aqueous Phase Systems, Applications In Cell Biology and Biotechnology, Plenum Press, N.Y. N.Y., 1989 pp. 211-213.

The present invention also relates to method of stimulating an immune response in a subject. Such a method can be performed, for example, by administering a composition of the invention, particularly an immunogenic peptide representative of a structural element of a target pathogen or pathogen protein product, or a polynucleotide encoding the composition, to a subject under conditions suitable for stimulating an immune response. Such conditions will depend, for example, on the material being administered. Where an immunogenic peptide is administered, it can be administered, for example, subcutaneously, subdermally, mucosally or intravenously and can, but need not be formulated with an adjuvant, and can be administered as an initial dose and in booster doses, either or any of which can include an adjuvant.

A subject treated according to a method of the invention can be any subject in which it is desired to stimulate an immune response using an immunogenic peptide of the invention, e.g., inducing an immune response in a subject to reduce or lower the risk of infection upon exposure to the pathogen or pathogen protein product, e.g., anthrax PA. As such, the subject can be a vertebrate subject, including, for example, a mammalian subject such as a rabbit, goat, mouse, or other mammal, thus providing a means to generate antibodies specific for the target protein for which the immunogenic peptide represents a structural element. Accordingly, the method can further include isolating such antibodies from the mammalian subject. In one embodiment, the subject treated according to a method of the invention is a human subject, wherein the immune response in the subject can be stimulated to protect the subject from harm due to an infectious microorganism that expresses the target protein, or to ameliorate such harm following infection with the microorganism. In one aspect, the subject is a human subject infected with or susceptible to infection with B. anthracis, and administration of the immunogenic peptide generates a protective immune response in the subject against the signs and symptoms of anthrax.

In another embodiment of the invention, methods are provided herein of making pharmaceutical compositions of antibody fragments or functional fragments thereof as therapeutic agents. The productions of antibody fragments or functional fragments thereof produced from monoclonal antibodies are well known to those skilled in the art (Kohler, et al., Nature, 256:495, 1975). For example, fragments may include, but are not limited to, Fab and F(ab′)2, Fv and SCA fragments such as scFv and scAb, which are capable of binding an epitopic determinant on a protein of interest.

The invention describes antibody fragments or functional fragments thereof that do not contain the constant antibody region, or the Fc fragment. Whole antibodies are typically costly and labor intensive to produce. In contrast, antibody fragments or functional fragments thereof, with their reduced size, can be produced in a bacterial expression system at a reduced cost as compared to production of whole antibodies, e.g., the cost can be about 1/50, 1/100 or 1/200 of the cost per molecule produced.

The antibody fragments or functional fragments thereof described herein can also include chimeric antibody fragments or functional fragments thereof, including the products of a Fab or other immunoglobulin expression library. These antibody fragments or functional fragments thereof retain the ability to selectively bind with its antigen or receptor and are defined as follows: (1) Fab, the fragment which contains a monovalent antigen-binding fragment of an antibody molecule can be produced by digestion of whole antibody with the enzyme papain to yield an intact light chain and a portion of one heavy chain; (2) Fab′, the fragment of an antibody molecule can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), defined as a genetically engineered molecule containing the variable region of the light chain, the variable region of the heavy chain, and linked by a suitable polypeptide linker as a genetically fused single chain molecule. For example, any of the above fragments can be single chain antibody molecules, including scFv and scAb.

Methods of making these fragments are known in the art. See, for example, Harlow and Lane, Antibodies: A Laboratory Manual, Cold Spring Harbor Laboratory, New York, 1988, incorporated herein by reference.

A skilled artisan will be able to determine suitable variants of the polypeptide and functional fragment thereof as set forth herein using well-known techniques. In certain embodiments, one skilled in the art may identify suitable areas of the polypeptide that may be changed without destroying activity by targeting regions not believed to be important for activity. In other embodiments, the skilled artisan can identify residues and portions of the polypeptides that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three dimensional structure. In certain embodiments, one skilled in the art may choose to not make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult, 1996, Curr. Op. in Biotech. 7:422-427; Chou et al, 1974, Biochemistry 13:222-245; Chou et al, 1974, Biochemistry 113:211-222; Chou et al, 1978, Adv. Enzymol Relat. Areas Mol. Biol. 47:45-148; Chou et al, 1979, Ann. Rev. Biochem. 47:251-276; and Chou et al, 1979, Biophys. J. 26:367-384. Moreover, computer programs are currently available to assist with predicting secondary structure. One method of predicting secondary structure is based upon homology modeling. For example, two polypeptides or proteins that have a sequence identity of greater than about 30%, or similarity greater than 40% often have similar structural topologies. The recent growth of the protein structural database has provided enhanced predictability of secondary structure, including the potential number of folds within a polypeptide's or protein's structure. See Holm et al, 1999, Nucl. Acid. Res. 27:244-247. It has been suggested (Brenner et al, 1997, Curr. Op. Struct. Biol. 7:369-376) that there are a limited number of folds in a given polypeptide or protein and that once a critical number of 5 structures have been resolved, structural prediction will become dramatically more accurate.

Additional methods of predicting secondary structure include “threading” (Jones, 1997, Curr. Opin. Struct. Biol. 7:377-87; Sippl et al, 1996, Structure 4:15-19), “profile analysis” (Bowie et al, 1991, Science 253:164-170; Gribskov et al, 1990, Meth. Enzym. 183:146-159; Gribskov et al, 1987, Proc. Nat. Acad. Sci. 84:4355-4358), and “evolutionary linkage” (See Holm, 1999, supra; and Brenner, 1997, supra).

The present invention also relates to antibody fragment production in a vector expression system, where the polynucleotide encodes a peptide, or an antibody fragment, for example, a peptide portion of a pathogen or pathogen protein product and the coding sequence generally is contained in a vector and is operatively linked to elements at the 5′ or 3′ terminus of the polynucleotide, for example, operatively linked to appropriate regulatory elements, including, if desired, a tissue specific promoter or enhancer; or

The vector can be a cloning vector, which is useful for maintaining the polynucleotide, or can be an expression vector, which contains, in addition to the polynucleotide, regulatory elements useful for expressing the polynucleotide and, where the polynucleotide encodes a peptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. Although, the vector described herein is for use in a prokaryotic system, the vector also can contain elements required for replication in either a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993; each of which is incorporated herein by reference).

The encoded peptide can be further operatively linked, for example, to peptide tag such as a His-4, His-6, His-8 or more and the like, which can facilitate identification of expression of the agent in the target cell. A polyhistidine tag peptide such as His-6 can be detected using a divalent cation such as nickel ion, cobalt ion, or the like. Additional peptide tags include, for example, a FLAG epitope, which can be detected using an anti-FLAG antibody (see, for example, Hopp et al., BioTechnology 6:1204 (1988); U.S. Pat. No. 5,011,912, each of which is incorporated herein by reference); a c-myc epitope, which can be detected using an antibody specific for the epitope; biotin, which can be detected using streptavidin or avidin; and glutathione S-transferase, which can be detected using glutathione. Such tags can provide the additional advantage that they can facilitate isolation of the operatively linked peptide or peptide agent, for example, where it is desired to obtain a substantially purified peptide corresponding to a proteolytic fragment of a myostatin polypeptide.

The polynucleotide, which can be contained in a vector, can be introduced into a cell by any of a variety of methods known in the art (Sambrook et al., Molecular Cloning: A laboratory manual (Cold Spring Harbor Laboratory Press 1989); Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell, for example bacteria versus yeast, into which the polynucleotide is to be introduced; as well as whether the cell is isolated in culture, or is in a tissue or organ in culture or in situ.

The antibody fragments or functional fragments thereof described herein function by binding, or specifically binding, or binds specifically to various and specific epitopes. As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. The term “binds specifically” or “specific binding activity,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)₂, Fd, Fv, scAb, and scFv fragments of an antibody that retain specific binding activity for an epitope of a anthrax protective antigen (anthrax-PA), are included within the definition of an antibody.

If desired, a kit incorporating an antibody or other agent useful in a method of the invention can be prepared. Such a kit can contain, in addition to the agent, a pharmaceutical composition in which the agent can be reconstituted for administration to a subject. The kit also can contain, for example, reagents for making the PEG-modified antibody fragment, and/or use of the antibody fragment for direct administration to a subject. Such reagents useful for covalently linking the antibody fragment to PEG or another polymer are described herein and known in the art.

Although the antibody fragments or functional fragments thereof described herein are expressed in a recombinant expression vector system, other methods for raising antibodies, for example, in a rabbit, goat, mouse or other mammal, are within the scope of the invention, so long as the antibody fragment does not contain an Fc region or fragment. Well known methods in the art include, for example, Green et al., “Production of Polyclonal Antisera,” in Immunochemical Protocols (Manson, ed., Humana Press 1992), pages 1-5; Coligan et al., “Production of Polyclonal Antisera in Rabbits, Rats, Mice and Hamsters,” in Curr. Protocols Immunol. (1992), section 2.4.1; each or which is incorporated herein by reference). In addition, monoclonal antibodies can be obtained using methods that are well known and routine in the art (Harlow and Lane, supra, 1988). The monoclonal antibodies can be further screened for the inability to bind specifically with the anthrax-PA. Such antibodies are useful, for example, for preparing standardized kits for clinical use. A recombinant phage that expresses, for example, a single subunit of the antibody fragment is also suggested in the present invention.

Although the invention describes antibody production or expression in E. coli of polynucleotide encoding the antibody fragment, the antibody fragments or functional fragments thereof can also be prepared by proteolytic hydrolysis of the antibody. Antibody fragments or functional fragments thereof can be obtained by pepsin or papain digestion of whole antibodies by conventional methods. For example, antibody fragments or functional fragments thereof can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, for example, Goldenberg, U.S. Pat. No. 4,036,945 and U.S. Pat. No. 4,331,647, each of which is incorporated by reference, and references contained therein; Nisonhoff et al., Arch. Biochem. Biophys. 89:230. 1960; Porter, Biochem. J. 73:119, 1959; Edelman et al., Meth. Enzymol., 1:422 (Academic Press 1967), each of which is incorporated herein by reference; see, also, Coligan et al., supra, 1992, see sections 2.8.1-2.8.10 and 2.10.1-2.10.4).

Other methods of cleaving antibodies, such as separation of heavy chains to form monovalent light/heavy chain fragments, further cleavage of fragments, or other enzymatic, chemical, or genetic techniques can also be used, provided the fragments specifically bind to the antigen that is recognized by the intact antibody. For example, Fv fragments comprise an association of V_{H} and V_{L} chains. This association can be noncovalent (Inbar et al., Proc. Natl. Acad. Sci., USA 69:2659, 1972). Alternatively, the variable chains can be linked by an intermolecular disulfide bond or cross-linked by chemicals such as glutaraldehyde (Sandhu, supra, 1992). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., Methods: A Companion to Methods in Enzymology 2:97, 1991; Bird et al., Science 242:423-426, 1988; Ladner et al., U.S. Pat. No. 4,946,778; Pack et al., Bio/Technology 11: 1271-1277, 1993; each of which is incorporated herein by reference; see, also Sandhu, supra, 1992.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106, 1991, which is incorporated herein by reference).

Variants useful for the present invention comprise various mutants and recombinants produced in bacteria, for example, any clone from an expression library, analogs, homologs, muteins and mimetics of a anthrax-PA antibody fragment that retain the ability to specifically bind to their respective antigens. The variants can be generated directly from anthrax-PA antibody fragment itself by chemical modification, by proteolytic enzyme digestion, or by combinations thereof. Additionally, genetic engineering techniques, as well as methods of synthesizing polypeptides directly from amino acid residues, can be employed.

The recombinant polypeptides or antibody fragments or functional fragments thereof expressed using the methods described herein are isolated and purified by conventional procedures, including separating the cells from the medium by centrifugation or filtration, precipitating the proteinaceous components of the supernatant or filtrate by means of a salt, e.g. ammonium sulfate, purification by a variety of chromatographic procedures, e.g. ion exchange chromatography or affinity chromatography, or the like. Methods of protein purification are known in the art (see generally, Scopes, R., Protein Purification, Springer-Verlag, N.Y. (1982), which is incorporated herein by reference) and may be applied to the purification of the recombinant proteins of the present invention.

Still, methods of synergistic therapy are encompassed by the present invention. For example, antibody fragments or functional fragments thereof described herein can be used in synergistic combination with sub-inhibitory concentrations of antibiotics. Examples of particular classes of antibiotics useful for synergistic therapy with the peptides of the invention include aminoglycosides (e.g., tobramycin), penicillins (e.g., piperacillin), cephalosporins (e.g., ceftazidime), fluoroquinolones (e.g., ciprofloxacin), carbapenems (e.g., imipenem), tetracyclines and macrolides (e.g., erythromycin and clarithromycin). Further to the antibiotics listed above, typical antibiotics include aminoglycosides (amikacin, gentamicin, kanamycin, netilmicin, tobramycin, streptomycin, azithromycin, clarithromycin, erythromycin, erythromycin estolate/ethylsuccinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, cefamandole, cefoxitin, cefuroxime, cefonicid, cefinetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin). Other classes of antibiotics include carbapenems (e.g., imipenem), monobactams (e.g., aztreonam), quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), and glycopeptides (e.g., vancomycin, teicoplanin), for example. Other antibiotics include chloramphenicol, clindamycin, trimethoprim, sulfamethoxazole, nitrofurantoin, rifampin, mupirocin and the like.

Further, it will be understood by one skilled in the art that the specific dose level and frequency of dosage for any particular subject in need of treatment may be varied and will depend upon a variety of factors. These factors include the activity of the specific polypeptide or functional fragment thereof, or the metabolic stability and length of action of that compound, the age, body weight, general health, sex, diet, mode and time of administration, rate of excretion, drug combination, the severity of the particular condition, and the host undergoing therapy. Generally, however, dosage will approximate that which is typical for known methods of administration of the specific compound. Also, dosage may vary depending on the route of administration, e.g., a subcutaneous administration the antibody fragment or functional fragments thereof and pharmaceutical carrier or exipient to a human subject, an appropriate amount may be more about than for other forms of administration. Hence, an appropriate amount can be determined by one of ordinary skill in the art and using routine procedures as discussed above.

The compositions and formulations of the invention can be administered systemically or locally. The antibody fragment of the invention can be administered parenterally by injection or by gradual infusion over time. The antibody fragment can be administered intravenously, intraperitoneally, intramuscularly, subcutaneously, intracavity, or transdermally. Other methods for delivery of the antibody fragment include orally, by encapsulation in microspheres or proteinoids, by aerosol delivery to the lungs, or transdermally by iontophoresis or transdermal electroporation. Other methods of administration will be known to those skilled in the art. In a preferred embodiment, the antibody fragment is administered subcutaneously.

Preparations for parenteral administration of a antibody fragment of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The invention also relates to pharmaceutical compositions, which compositions can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The local composition format can be selected from the group consisting of an aerosol (e.g., nebulization, dry powder or metered dose inhalation), a drop, a spray, a cream, and an ointment. Also, depending on the format, the compositions can include other carrier agents including swelling mucoadhesive particulates, pH sensitive microparticulates, nanoparticles/latex systems, ion-exchange resins and other polymeric gels and agents (Ocusert, Alza Corp., California; Joshi, A., S. Ping and K. J. Himmelstein, Patent Application WO 91/19481). These agents and systems maintain prolonged drug contact with the absorptive surface preventing washout and nonproductive drug loss.

The compositions of the invention can also have formulations whereby the antibody fragments or functional fragments thereof are in a delayed-released format. Suitable examples of preparations having a delayed release are, for example, semi-permeable matrices consisting of solid hydrophobic polymers which contain the protein; these matrices are shaped articles, for example film tablets or microcapsules. Examples of matrices having a delayed release are polyesters, hydrogels [e.g. poly(2-hydroxyethyl methacrylate)—described by Langer et al., J. Biomed. Mater. Res., 15:167-277 [1981] and Langer, Chem. Tech., 12:98-105 [1982]—or poly(vinyl alcohol)], polyactides (U.S. Pat. No. 3,773,919, EP 58,481), copolymers of L-glutamic acid and gamma-ethyl-L-glutamate (Sidman et al., Biopolymers, 22:547-556 [1983]), non-degradable ethylene/vinyl acetate (Langer et al., loc. sit.), degradable lactic acid/glycolic acid copolymers such as Lupron Depot™ (injectable microspheres consisting of lactic acid/glycolic acid copolymer and leuprolide acetate) and poly-D-(−)-3-hydroxybutyric acid (EP 133,988). While polymers such as ethylene/vinyl acetate and lactic acid/glycolic acid enable the molecules to be released for periods of greater than about 100 days, the proteins are released over relatively short periods of time in the case of some hydrogels. If encapsulated proteins remain in the body over relatively long periods of time, they can then be denatured or aggregated by moisture at 37° C., resulting in a loss of biological activity and possible changes in immunogenicity. Meaningful strategies for stabilizing the proteins can be developed, depending on the mechanism involved. If it is found, for example, that the mechanism which leads to the aggregation is based on intermolecular S—S bridge formation as a result of thiodisulphide exchange, stabilization can be achieved by modifying the sulphydryl radicals, lyophilizing from acid solutions, controlling the moisture content, using suitable additives and developing special polymer/matrix compositions.

The formulations of the invention exhibiting delayed release can be optionally enclosed in liposomes prepared by methods which are known in the art. See DE 3,218,121; Epstein et al., Proc. Natl. Acad. Sci. USA, 82;3688-3692 (1985); Hwang et al., Proc. Natl. Acad. Sci. USA, 77:40304034 (1980); EP 52,322; EP 36,676; EP 88,046; EP 143,949; EP 142,641; Japanese Patent Application 83-118008; U.S. Pat. Nos. 4,485,045 and 4,544,545; and also EP 102,324.

Also contemplated is use of the DNA sequences encoding the antibody fragment or functional fragments thereof of this invention in gene therapy applications (in vivo and ex vivo). Gene therapy applications contemplated include treatment of those diseases in the antibody fragment or functional fragments thereof is expected to provide an effective therapy, e.g., as an antagonist, due to its immunomodulatory activity,

Local the antibody fragment or functional fragments thereof using gene therapy may provide the therapeutic agent to the target area. Both in vitro and in vivo gene therapy methodologies are contemplated. Several methods for transferring potentially therapeutic genes to defined cell populations are known. See, e.g., Mulligan, “The Basic Science Of Gene Therapy”, Science, 260: 926-31 (1993). These methods include: 1) Direct gene transfer. See, e.g., Wolff et al, “Direct Gene transfer Into Mouse Muscle In Vivo”, Science. 247:1465-68 (1990); 2) Liposome-mediated DNA transfer. See, e.g., Caplen at al., “Liposome-mediated CFTR Gene Transfer To The Nasal Epithelium Of Patients With Cystic Fibrosis”, Nature Med. 3: 39-46 (1995); Crystal, “The Gene As A Drug”, Nature Med. 1:15-17 (1995); Gao and Huang, “A Novel Cationic Liposome Reagent For Efficient Transfection Of Mammalian Cells”, Biochem. Biophys. Res. Comm., 179:280-85 (1991); 3) Retrovirus-mediated DNA transfer. See, e.g., Kay et al., “In Vivo Gene Therapy Of Hemophilia B: Sustained Partial Correction In Factor IX-Deficient Dogs”, Science, 262:117-19 (1993); Anderson, “Human Gene Therapy”, Science 256:808-13 (1992). 4) DNA Virus-mediated DNA transfer. Such DNA viruses include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al., “The Use Of DNA Viruses As Vectors For Gene Therapy”, Gene Therapy, 1:367-84 (1994); U.S. Pat. No. 4,797,368, incorporated herein by reference, and U.S. Pat. No. 5,139,941, incorporated herein by reference; and 5) Ex vivo gene therapy.

In another aspect of the invention, methods are provided for ex vivo gene therapy, in which recombinant vectors are introduced into stem cells or animal cells (e.g., human), or other accessible cell population, and, after engineering and propagation, are transplanted back into the patient. Such engineered constructs might be designed to produce RNA that will be translated into antibody fragments or functional fragment proteins, or altered (mutated) versions thereof. Alternatively, such constructs might be designed to produce antisense RNA designed to inhibit transcription or translation, or to produce ribozymes that target pathogens or pathogen protein products for degradation.

These methods are known to those in the art, and may include stable integration of DNA sequences by recombination, adenoviral, retroviral and other means which are intended to introduce and propagate sequences in engineered cells, by techniques such as pronuclear microinjection, liposome mediated uptake, electroporation of embryos, homologous (targeted) recombination, and so forth. One embodiment is receptor-mediated gene transfer, whereby the transgene is coupled with a ligand via polylysine, where the ligand is some molecule that interacts selectively with surface molecules, or receptors, on a selective cell population or specificity for a cell-specific surface marker. Depending on the method chosen, the introduced gene might replicate autonomously as part of a vector, or may integrate a specific or random sites. Such a gene may be engineered so as to contain regulatory sequences that drive expression in a constitutive, inducible, tissue- or cell-cycle specific, or other manner, as desired.

Other embodiments, although not described in detail, are contemplated and are encompassed by the invention, for example, the invention incorporates herein by reference in their entirety Harvey et al. (2004) “Anchored periplasmic expression, a versatile technology for the isolation of high-affinity antibodies from Escherichia coli-expressed libraries,” PNAS, 101(25):9193-9198; WO03040384 to Georgiou et al. (published May 15, 2003) “Recombinant antibodies for the detection and neutralization of anthrax toxin”; and Mabry R. et al. (2005) “Passive protection against anthrax by using a high affinity antitoxin antibody fragment lacking an Fc region,” Infection and Immunity, in press.

Materials and Methods

Bacterial cell culture conditions. B. anthracis strain Vollum 1B was originally acquired from USAMRIID, and all manipulations of this organism were performed under BSL-3 and BSL-4 biocontainment. A single colony was subcultured in 4 mL of nutrient broth (Difco) at 37° C. for 48 h prior to inoculation (1 mL each) into four baffled spinner flasks containing 250 mL of nutrient broth medium. After a 48-h incubation, cells were pelleted by centrifugation at 1,500×g for 10 min, resuspended into 20 mL G medium (Goodson, R. J., and N. V. Katre (1990) Biotechnology (NY) 8:343-346), and transferred into a 1-liter aerated vessel containing 500 mL G medium. Following 72 h of incubation at 37 C, cells were pelleted as above, washed briefly with sterile water, resuspended in 20 mL G medium containing 10% glycerol, and stored at −80 C. Aliquots (1 mL) were serially diluted, and one-half of each dilution was subjected to 10 min of heat treatment at 75° C. to confirm spore conversion. Both heat-treated and nonheat-treated dilutions were plated in duplicate on sheep blood agar plates to determine spore concentrations.

scAb expression and purification. The M18 scFv gene (Harvey et al. (2004) Proc. Natl. Acad. Sci. USA 101:9193-9198) was cloned via terminal SfiI sites into pMoPac16 (Hayhurst et al. (2003) J. Immunol. Methods 276:185-196), a modified version of the pAk4000 vector (Krebber et al. (1997) J. Immunol. Methods 201:35-55) that carries an scFv gene with the human K light chain to create a single-chain antibody (scAb) (Hayhurst et al. (2003); McGregor et al. (1994) Mol. Immunol. 31:219-226). The pMoPac16 plasmid also carries a gene for the coexpression of the skp periplasmic chaperone (Hayhurst et al. (2003); Hayhurst et al. (1999) Protein Expr. Purif. 15:336-343). To create a protein suitable for PEG conjugation, the M18 scAb gene was amplified with a C-terminal primer that incorporated a Cys residue downstream of the C-terminal His6 purification tag. This latter gene was then ligated into pMoPac 16 via NcoI and AscI restriction sites to create the pMoPac16Cys plasmid that contains the M18 scAb-His6-Cys construct.

Escherichia coli Tuner cells (Novagen, Madison, Wis.) were transformed with pMoPac16_M18 and pMoPac16Cys_M18 and were grown at 25° C. in 2-liter baffled flasks containing 400 mL TB medium with 2% glucose and 200 μg/mL ampicillin. Cultures were induced at an optical density at 600 nm of 1.6 with 1 mM IPTG (isopropyl-p-D-thiogalactopyranoside) (Sigma-Aldrich, St. Louis, Mo.) for 4 h, and then the cells were pelleted by centrifugation (10 min at 8,000×g). Osmotic shock was carried out at 0° C. as previously described (Hayhurst et al. (2003). Cells were resuspended in 20 mL 0.75 M sucrose and 100 mM Tris-HCl (pH 8.0), with the addition of 1.0 mL of 10 mg/mL lysozyme in the same buffer. After shaking for 10 min at 0° C., 40 mL of 1 mM EDTA was added drop wise, followed by 15 min of further incubation at 0° C. A total of 3.0 mL of 0.5 M MgCl2 was then added drop wise, followed by an additional 15 min of incubation at 0° C. Spheroplasts were pelleted by centrifugation, and the clarified supernatant was mixed with a 1/10 volume of 10× IMAC buffer (100 mM Tris-HCl, 5 M NaCl, 0.2 M imidazole, pH 8.0) and applied to 1.5 mL Ni-nitrilotriacetic acid agarose resin (QIAGEN, Madison, Wis.). Following washing with 3×10 mL IMAC buffer, scAb protein was eluted with 500 mM imidazole in IMAC buffer and dialyzed against 2×2 liters phosphate-buffered saline (PBS) at 4° C. overnight. Native M18 scAb was then applied to a Superdex 200 HR10/30 column (Amersham Biosciences, Piscataway, N.J.) on an Akta fast-protein liquid chromatography system (Amersham Pharmacia, Piscataway, N.J.). Fractions were isolated and then concentrated with an Amicon ultra centrifugal filter device (molecular weight cutoff, 10; Millipore Corp., Bedford, Mass.). Protein concentrations were quantified using a micro-bicinchoninic acid quantification kit (Pierce, Rockford, Ill.).

ScAb conjugation and purification. The conjugation of M18 scAb with PEG was performed as described previously (Pannifer et al. (2001) Nature 414:229-233) with the following modifications: a twofold excess of tris(2-carboxyethyl)phosphine hydrochloride (TCEP; Molecular Probes, Eugene, Oreg.) in PBS was added to purified scAb protein (2 mg/mL in PBS) and incubated overnight, with stirring at 4° C. The solution was then brought to room temperature and a twofold excess of maleimide-PEG 20 kDa or 40 kDa (Nektar Therapeutics, San Carlos, Calif.) in PBS was added drop wise over 2 to 4 h. The scAb-PEG conjugate was purified by IMAC using Ni-nitrilotriacetic acid agarose. Eluate was then purified by size exclusion chromatography, concentrated, and quantified as mentioned for native scAb.

Biacore analysis. Surface plasmon resonance (SPR) analysis was performed using a Biacore 3000 instrument (Biacore, Piscataway, N.J.). Recombinant PA (List Biological Laboratories, Campbell, Calif.) in 10 mM NaC2H3O2 (pH 5.0) was immobilized on a CM5 chip using 1-ethyl-3-(3-dimethylaminopropyl)carbodiimide N-hydroxy-succinimide chemistry. The PA solution was added as a 5 ug/mL solution until an amount equivalent to 500 response units was obtained. ScAb and scAb-PEG proteins (2.5 nM to 40 nM) in HBS-N (Biacore) were used at a flow rate of 100 μl/min. A solution of 4 M MgCl2 was used as the regeneration buffer between runs. Data were analyzed using BIAevaluation software (version 3.0). A second flow cell with bovine serum albumin was used for data baseline correction. For some experiments, the scAb with or without PEG protein was immobilized onto the chip at a level corresponding to 120 response units under the same coupling conditions as above and PA was used as the analyte.

In vitro anthrax toxin challenge. Anthrax LeTx challenges were performed with RAW 264.7 mouse macrophage cells as previously described (Kitamura et al. (1990) Biochem Biophys Res Commun. 171(3):1387-94; and Varughese et al. (1998) Internalization of a Bacillus Mol Med. 4, 87-95).

Pharmacokinetic studies. Female Hartley guinea pigs (225 to 305 g) (Charles River Laboratories, Wilmington, Mass.) were given dorsal, subcutaneous injections of 1.2 mL of M18 scAb-PEG protein (20 or 40 kDa) or 14B7 IgG to a dose of 10 mg/kg or PBS as a control. Following sedation with ketamine (80 mg/kg) and xylazine (10 mg/kg), animals were bled when time reached 0 for 15 min, 1 h, 3 h, 6 h, 12 h, 24 h, 48 h, and 72 h at the femoral artery. The serum was collected and centrifuged as mentioned above. The construct concentrations were determined by enzyme-linked immunosorbent assay as follows: PA (2.5 ug/mL) in PBS was coated onto CoStar 96-well plates (Corning Inc., Corning, N.Y.) and incubated overnight at 4° C. The wells were then blocked with the addition of 2% milk-PBS for 3 h at room temperature, and diluted serum (1:10) was added in 2% milk-PBS. ScAb-PEG constructs were detected by using goat anti-K light chain horseradish peroxidase (Sigma-Aldrich, St. Louis, Mo.). 14B7 murine IgG was detected with goat anti-mouse IgG horseradish peroxidase (Bio-Rad, CA). Data were modeled using WinNonlin software (Pharsight, Mountain View, Calif.) formatted to be consistent with a one-compartment, bolus, first-order elimination model.

Inhalation challenge with B. anthracis spores. Animals were housed individually in a One Cage 2100 AllerZone interchangeable microisolator high density housing system (Lab Products Inc., Seaford, Del.) in the BSL-4 safety facility at the Southwest Foundation for Biomedical Research (San Antonio, Tex.). Anthrax spore inocula were prepared on the day of challenge and diluted to the desired concentration in PBS. Female Hartley guinea pigs (225 to 305 g) were sedated with ketamine (80 mg/kg) and xylazine (10 mg/kg) during all bleeds, injections, and inhalation instillations. Animals were injected subcutaneously with 3.0 mL of PBS containing M18 scAb-PEG 40 kDa to a dosage of 40 mg/kg or 80 mg/kg of body weight. Control animals were injected with 3.0 mL of either PBS, unconjugated 40-kDa PEG-maleimide (40 mg/kg), or native M18 scAb (40 mg/kg). Four hours later, the animals were challenged with anthrax spores by unilateral instillation of 250× to 625×LD50 (1×107 to 2.5×107) of B. anthracis Vollum 1B (Altboum, et al. (2002) Infect. Immun. 70:6231-6241) spore inocula (100 μL total volume) in the nares of animals. Animals were monitored for a 2-week period and euthanized by cardiac injection of sodium pentobarbital when considered moribund or at project end.

Lungs and spleens were removed from the guinea pigs after euthanasia. A 100-mg section was excised and homogenized with 0.4 mL of sterile PBS in a sterile tissue grinder. The homogenates were serially diluted in sterile PBS and then plated in duplicate on sheep blood agar plates. After 24 h of incubation at 37° C., the colonies were enumerated to determine CFU/100 mg of tissue.

The following examples are intended to illustrate but not limit the invention.

### EXAMPLE 1

### Conjugate Preparation, Expression and Characterization of the scAB Form of a Protective Antibody Fragment

This example describes methods for preparing a polynucleotide encoding a recombinant scAb, expression of the recombinant polynucleotide, and characterization of the scAb polypeptide, which is protective against the pathogenic challenge.

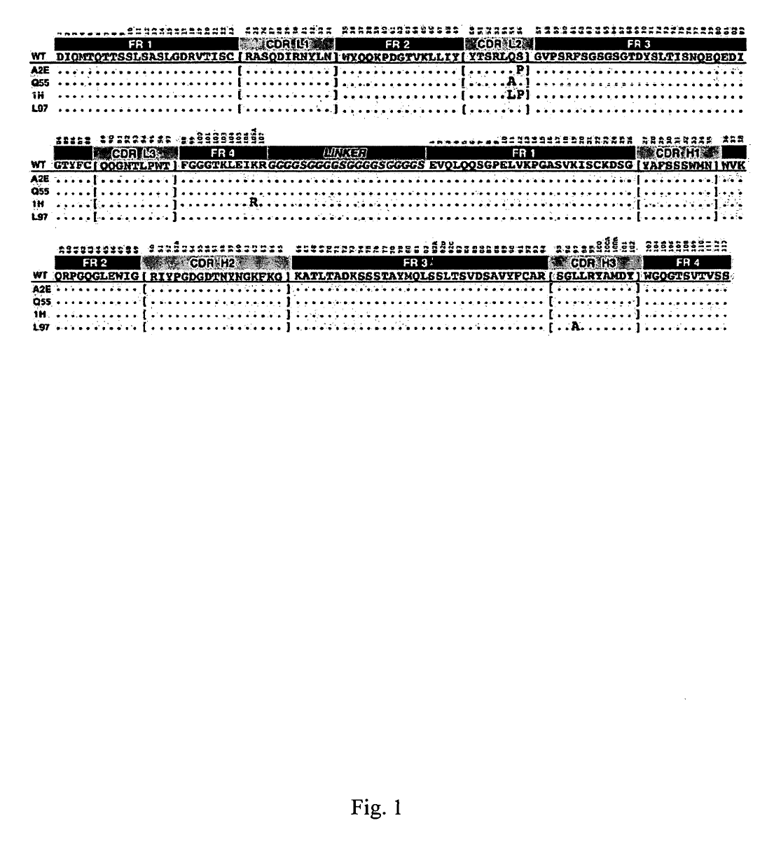

Briefly, various clones, including M18, were obtained by mutagenized by error-prone PCR the gene encoding the anti-PA 14B7 scFv (FIG. 1) and fused to the E. coli NIpA anchoring sequence in an appropriate expression vector and the resulting library was transformed in E. coli (Harvey et al (2004), PNAS USA 101:9193-9198). Nucleotide sequence analysis of the clones selected at random had an average of about 2% nucleotide substitutions per gene.

The scFv DNA from the second round was subsequently amplified using PCR and ligated into pMoPac16 expression vector. The expression of the antibody protein was in the scAb format and is comprised of a scFv whereby the light chain was fused to a human κ constant region. See Harvey et al (2004); and McGregor, et al. (1994) Mol. Immunol. 31:219-226. Selected colonies were chosen and induced with IPTG to express the protein. Three scAb proteins (or clones) with the slowest antigen disassociation kinetics were produced in large scale and purified (see Table I below). The three scAbs were: M5 (7 amino acid substitutions); M6 (12 amino acid substitutions); and M18 (11 amino acid substitutions). As shown in Table I, M18 is the highest affinity clone and exhibiting a K_{D} of about 35 pM. In another instance, M18 had a K_{D} of 54 pM, which was about 5-fold improved over the whole antibody to PA. M18 contains the S56P mutation and lacks the Q55L substitutions found in M5 and M6. The introduction of the Q55L substitution into M18 improved antigen binding. The M18 scAb fragment includes mutations I12V, L46F, S56P, S76N, Q78L, and L94P on the light chain, and S30N, T57S, K64E, and T681 on the heavy chain.

TABLE I
Affinity Data Acquired by Surface Plasmon Resonance (SPR)

| | K_{D} | Kₒₙ | K_{off} | |
|---|---|---|---|---|
| Antibody | (pM) | (1/Ms) | (1/s) | Mutations |
| 14B7 | 4300 | 7.1 × 10⁵ | 3.0 × 10⁻³ | |
| M5 | 96 | 1.1 × 10⁶ | 1.1 × 10⁻⁴ | Light: Q38R, Q55L, |
| | | | | S56P, T74A, Q78L |
| | | | | Heavy: K62R |
| M6 | 69 | 1.2 × 10⁶ | 8.2 × 10⁻⁵ | Light: S22G, L33S, |
| | | | | Q55L, S56P, Q78L, |
| | | | | L94P |
| | | | | Heavy: S7P, K19R, |
| | | | | S30N, T68I, M80L |
| M18 | 35 | 1.2 × 10⁶ | 4.2 × 10⁻⁵ | Light: I12V, L46F, |
| | | | | S56P, S76N, Q78L, |
| | | | | L94P |
| | | | | Heavy: S30N, T57S, |
| | | | | K64E, T68I |

The scAb form of M18 was fused to a His6 tag followed by a terminal Cys residue for the conjugation of PEG-maleimide (Albrecht, et al. (2004) Bioconjug. Chem. 15:16-26; Natarajan, et al. (2005) Bioconjug Chem. 16:113-121; and Yang, et al. (2003) Protein Eng. 16:761-770). The antibodies described herein have superior expression, stability, and serum half-lives compared to scFv fragments (Maynard, et al. (2002) Nat. Biotechnol. 20:597-601).

Expression from a lac promoter in E. coli Tuner cells, followed by purification by IMAC, produced a mixture of a monomer and a disulfide-linked dimer with an average yield of 8 mg/liter (1.3 mg/liter optical density at 600 nm) in shake flask culture. Reduction of the inter-scAb disulfide by using TCEP produced pure monomeric M18 scAb, which was conjugated to either 20-kDa or 40-kDa PEG-maleimide. Following gel filtration fast-F1 protein liquid chromatography, the purified protein conjugates were found to contain <2 ng lipopolysaccharide/mg of protein by using the QCL-1000 Limulus amoebocyte lysate assay kit (Bio-Whittaker, MD).

The antigen binding kinetics of PEGylated and free M18 scAb antibody fragments was evaluated by SPR on a Biacore 3000 instrument using two complementary assay formats. The measured dissociation rates (K_{D}) of the antigen-antibody complex were not affected by the conjugation of PEG, regardless of whether PA or the scAb was immobilized on the chip. On the other hand, the association rates (K_{A}) did depend on the assay format. Specifically, binding of M18-scAb constructs in the mobile phase to PA immobilized on the Biacore chip resulted in a reduction in the rate of complex formation as the attached PEG size increased comparable to that observed in earlier studies (Yang, et al. (2003) Protein Eng. 16:761-770) (Table II). However, when the M18-scAb antibody T1 constructs were attached to the Biacore chip and binding was measured with free PA in the mobile phase, the observed on-rates were no longer affected by the presence or the size of the PEG chain, and, thus, similar overall equilibrium dissociation constants were measured for all constructs.

TABLE II
Kinetic analysis of anti-PA scAb with
or without PEG binding using SPR

| Biocore | | Analysis value for: | | |
|---|---|---|---|---|
| Format* | Construct | K_{A} (1/Ms) | K_{D} (1/Ms) | K_{D} (M) |
| PA down; | M18 scAb | 1.4 × 10⁶ | 6.3 × 10⁻⁵ | 4.4 × 10⁻¹¹ |
| antibody | M18 scAb- | 1.0 × 10⁵ | 7.0 × 10⁻⁵ | 6.9 × 10⁻¹⁰ |
| analyte | PEG 20 kDa | | | |
| | M18 scAb- | 2.7 × 10⁴ | 7.2 × 10⁻⁵ | 2.6 × 10⁻⁹ |
| | PEG 40 kDa | | | |
| Antibody | M18 scAb | 9.3 × 10⁵ | 8.7 × 10⁻⁵ | 9.5 × 10⁻¹¹ |
| down; PA | M18 scAb- | 7.8 × 10⁵ | 2.0 × 10⁻⁵ | 2.5 × 10⁻¹¹ |
| analyte | PEG 20 kDa | | | |
| | M18 scAb- | 8.7 × 10⁵ | 3.8 × 10⁻⁵ | 4.3 × 10⁻¹¹ |
| | PEG 40 kDa | | | |

### EXAMPLE 2

### In Vitro Neutralization of Mouse Macrophages Using scAB Fragment

Previously, it was shown that anti-PA whole antibodies prevented infections in animals (e.g. mice and rabbits) exposed to live anthrax spores (see PCT application WO03/040384 to Georgiou et al., which is incorporated herein by reference in its entirety). This example describes that the scAb fragment neutralizes mouse macrophages in vitro.

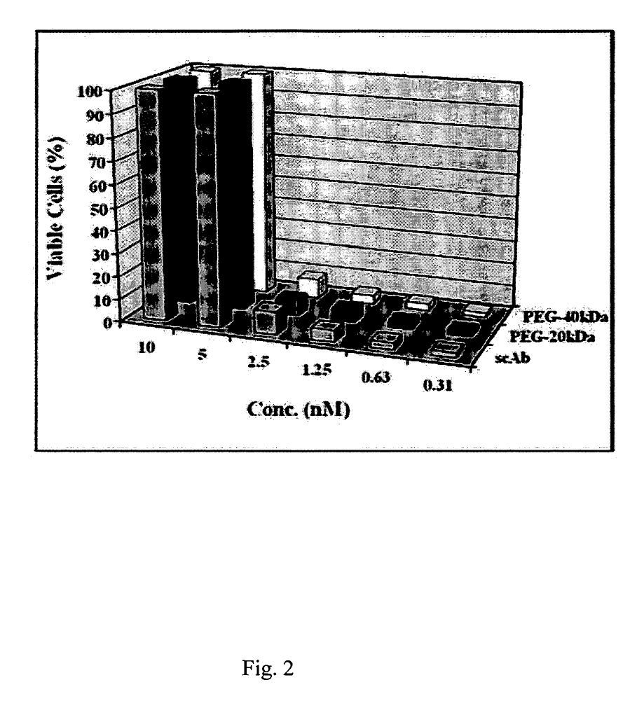

RAW 264.7 mouse macrophage cells were grown on microtiter wells, incubated with 0.31 to 10 nM scAb or PEGylated scAb as shown, and challenged 5 min later with lethal toxin (100 ng/mL PA, 80 ng/mL LF). The percentage of cells surviving toxin challenge at a specific antibody dose, compared to that of the negative control, untreated cells. The in vitro toxin neutralization activity of the M18 scAb-PEG 20 kDa and M18 scAb-PEG 40 kDa conjugates were analyzed using the mouse macrophage assay (Maynard, et al. (2002) Nat. Biotechnol. 20:597-601; and Singh, et al. (2001) J. Biol. Chem. 276:22090-94) (FIG. 2). The average of triplicate experiments is reported.

F2 RAW 264.7 mouse macrophage cells (ATCC TIB-71) were treated with M18 scAb, M18 scAb-PEG 20 kDa, and M18 scAb-PEG 40 kDa serially diluted from 10 nM to 0.31 nM. LeTx (100 ng/mL PA, 80 ng/mL LF) was added, and cell viability was assayed. The unconjugated M18 scAb, as well as both PEG conjugates, displayed complete protection at concentrations above 5 nM. These results verify that the presence of the PEG chain does not significantly influence neutralization of anthrax LeTx, consistent with the Biacore results.

### EXAMPLE 3

### The scAb Fragment is Protective Against In Vivo B. Anthracis Challenge Subcutaneously

This example describes the protective affect of scAb fragments against in vivo subcutaneous challenge of anthrax-PA in animals.

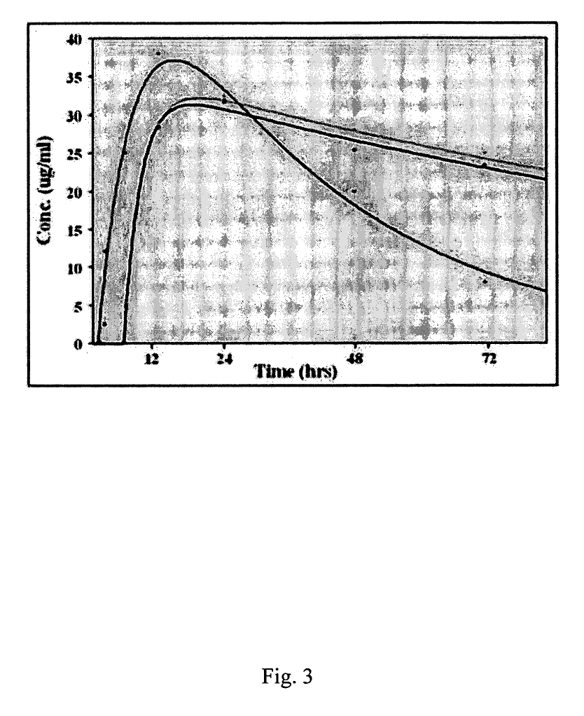

To evaluate the serum persistence of the M18 scAb-PEG antibody fragment, Hartley guinea pigs were injected subcutaneously with 10 mg/kg of M18 scAb-PEG 20 kDa, M18 scAb-PEG 40 kDa, or 14B7 murine monoclonal IgG. Serum concentrations of the constructs were determined by enzyme-linked immunosorbent assay, and the data were fit to a one-compartment, bolus, first-order elimination model (FIG. 3). Solid lines were calculated from a subcutaneous, bolus, first-order elimination model using WinNonLin software. As expected, scAb-PEG 20 kDa displayed a half-life of 23.2 h, 14B7 displayed a half-life of 96 h, and scAb-PEG 40 kDa displayed a half-life of 108 h. Hence, conjugation with PEG dramatically extended half-life in a manner dependent on the molecular mass of the PEG chain. Notably, the M18 scAb-PEG 40 kDa antibody conjugate displayed a serum half-life of 108 h, slightly longer than that of murine IgG (96 h).

### EXAMPLE 4

### The scAb Fragment is Protective Against In Vivo B. Anthracis Challenge by Inhalation

This example describes the protective effect of administering the scAb antibody post in vivo inhalation challenge.

Female Hartley guinea pigs (250 to 310 g) were exposed to 250× to 625×LD50 anthrax spores via intranasal inoculation 4 h after administration of scAb-PEG 40 kDa at 40 mg/kg (white triangles) or 80 mg/kg (grey squares), PBS (diamonds), unconjugated PEG-maleimide (square), or native scAb (dark square). Animals were monitored for at least 14 days after spore exposure.

Prophylactic treatment with M18 scAb PEG conjugates was tested in female Hartley guinea pigs challenged with B. anthracis Vollum 1B spores via the inhalation route. Guinea pigs were exposed to 250× to 625×LD50 spores of the Vollum 1B strain 3 h after subcutaneous injection of 40 mg/kg (five animals) and 80 mg/kg (six animals) of M18 scAb-PEG 40 kDa or controls using PBS (five animals) or unconjugated PEG (three animals) (FIG. 3). The relatively F4 high load of spores was intended to simulate a possible bioterrorism anthrax attack.

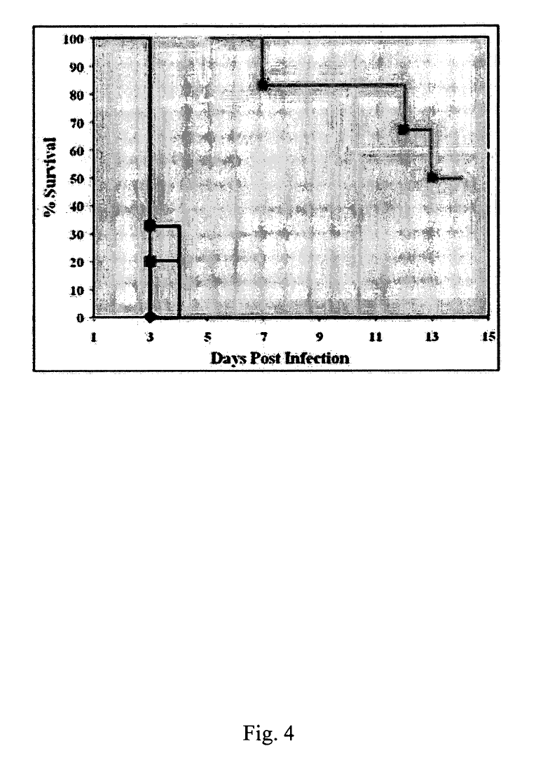

Within 72 hours, all animals treated with PBS alone or with unconjugated PEG died at times corresponding to dosage (Altboum, et al. (2002) Infect. Immun. 70:6231-41) with symptoms characteristic of inhalation anthrax toxemia (FIG. 4). Culture of tissues harvested at necropsy revealed high levels of B. anthracis in both the lungs (3.2×10⁷ to 1.6×10⁹ CFU/100 mg tissue) and spleen (1.3×10⁷ to 7.2×10⁹ CFU/100 mg tissue). In contrast, all 11 animals treated with a subcutaneous injection of M18 scAb-PEG 40 kDa exhibited significantly prolonged TTD, with 60% (⅗) and 50% ( 3/6) surviving the 14-day time course of the experiment at the 40 mg/kg and 80 mg/kg doses (P, <0.001; Holm-Sidak test), respectively (FIG. 4). Necropsy of the surviving animals that were euthanized at project end revealed healthy organ appearance, and no bacteria or spores were detected in the lungs or spleens in culture or upon microscopic examination of tissue samples. Analysis results of animals that perished after treatment with the 40 mg/kg dose displayed lymph depletion, edema, minimal hemorrhage, and low levels (about 10² to 10⁵ CFU/100 mg tissue) of anthrax bacteria in the lungs and spleens. Analysis of a guinea pig that succumbed in the 80 mg/kg antibody conjugate group showed a very small amount of hemorrhage and lymph depletion, yet no bacteria could be seen in a microscopic inspection of lung or spleen tissues.

PEG-scAb (M18) is injected subcutaneously into five adult guinea pigs 4 hours prior to exposure of the animals to about 100 to about 500 LD50's of live anthrax spores. A comparison of the pre-treated PEG-scAb (M18) animals versus the untreated PEG-scAb (M18), showed that PEG-scAb (M18) animals survived about 7 days longer. All untreated animals died within about 2-3 days of the injection, and autopsy reports reveal the expected internal anthrax lesions, particularly of the lungs. One PEG-scAb treated animal died, however, a preliminary autopsy indicates the cause of death in this animal may not be due to anthrax; rather it may be due to other natural or unnatural causes of death. The other four surviving animals will be monitored for 1-2 more weeks.

Discussion

The invention herein describes the ability of high-affinity anti-PA antibody fragments conjugated to PEG to confer prophylactic protection against challenge with inhalation anthrax spores. Previously, random mutagenesis and flow cytometric screening of a library derived from 14B7 scFv using APEx (Harvey et al (2004), PNAS USA 101:9193-9198) led to the isolation of the M18 antibody fragment that contains 10 amino acid substitutions (I21V, L46F, S56P, S76N, Q78L, and L94P of the light chain, and S30N, T57S, K64E, and T681 of the heavy chain based as set forth in SEQ ID NO: 1) and exhibits a 200-fold increase in affinity compared to that of 14B7 (Little et al. (1988) Infect. Immun. 56:1807-1813). The introduction of a single C-terminal Cys into the M18 scAb permitted conjugation to PEG-maleimide in a high yield without the formation of undesirable by-products such as antibody multimers or multiple PEG-conjugated species. The M18 antibody isolated by flow cytometric screening of microbial libraries (Chen et al. (2001) Nat. Biotechnology 19:537-542; Daugherty et al. (2000) Proc. Natl. Acad. Sci. USA 97:2029-2034) was well expressed. An average yield of about 8 mg/liter of shake flask culture was obtained using the moderately strong lac promoter. A satisfactory expression level is critical for the preparative production of antibody therapeutics.

Conjugation of PEG to M18 scAb had no effect on the dissociation rate constant, K_{D}, while the association rate constant, K_{A}, was found to be dependent on the Biacore assay format (Table II). When the PA was immobilized on the Biacore chip and the scAb constructs were in the mobile phase, a decrease in K_{A} with increasing PEG size was seen. No such trend was observed when the M18 scAb constructs were immobilized on the chip. It should be noted that the therapeutic potency of anti-PA antibody fragments can be influenced by the dissociation rate constant K_{D} (Maynard et al. (2002) Nat. Biotechnol. 20:597-601), which in the case of the M18 scAb, is not affected by PEGylation. Consistent with this assertion, PEGylation did not affect the activity of the antibody in protecting RAW 264.7 macrophages from toxin challenge in vitro. The PEG modification increased serum persistence to the point that following subcutaneous administration, the M18 scAb-PEG 40 kDa conjugate exhibited a serum half-life beyond that of the 14B7 whole IgG.

Prophylactic administration of the M18 scAb-PEG 40 kDa construct resulted in a significant increase in TTD and survival of about 50 to 60% of the animal group for the 14 day duration of the experiments compared to a 3-day mean TTD for the group of animals immunized with PBS alone, unconjugated PEG, or with native scAb. No statistically significant increase in protection was observed with the 80 mg/kg dose of M18 scAb-PEG 40 kDa compared to the 40 mg/kg dose, indicating that the latter or perhaps an even smaller dose may be adequate to confer the same level of protection.

In the group that was treated with 40 mg/kg, two of the six animals died on days 12 and 13. Yet, histological inspection of the surviving animals did not reveal any bacteria in the lungs or the spleens, suggesting that the infection had been eradicated.

The invention described herein demonstrate that an ultra high affinity, anti-PA antibody fragment lacking an Fc region is sufficient to confer prophylactic protection against challenge with inhalation anthrax spores. The prolonged TTD and low or nonexistent amount of live bacteria in all treated animals suggests that a higher degree of prophylaxis could be possible upon repeated antibody fragment administration.

The invention herein represents the first report of immunological protection against pathogen infection by antibody fragments lacking Fc domains and administered directly to the animal prior to challenge. In the absence of Fc, the mechanism of protection is unlikely to involve either antibody-dependent cytotoxicity or complement-dependent cytotoxicity. Thus, it appears that the interaction of the high affinity antibody fragment with toxin is sufficient to prevent the establishment of infection either by preventing spore germination (Collier et al. (2003) Cell Dev. Biol. 19:45-70), by preventing the dissemination of vegetative bacteria from lymphoid organs, or by perhaps some other mechanism.

Regardless of the mechanism, the extent of protection conferred by M18 scAb-PEG 40 kDa is roughly comparable to that observed with guinea pigs treated with rabbit polyclonal anti-PA IgG in a previous study (Little et al. (1997) Infect. Immun. 65:5171-5175). However, in the earlier study by Little et al. (Little et al. (1988) Infect. Immun. 56:1807-1813), the guinea pigs were exposed to a substantially lower dose of spores relative to the present invention (40×LD50 versus 250× to 600×LD50) and a different strain (Ames versus Vollum 1B), so that must be factored in when making any direct comparison. Nevertheless, it is reasonable to suggest that the protection observed with M18 scAb-PEG 40 kDa is significantly better than that reported for the parent 14B7 IgG monoclonal antibody (Little et al. (1997) Infect. Immun. 65:5171-5175). A reasonable conclusion is that the very high affinity of the recombinant M18 antibody fragment is responsible for the increased protective activity. Supporting the results described herein, another report noted that the interaction between PA and the CMG2 receptor is relatively strong; K_{D} was 170 pM (Wigelsworth et al. (2004) J. Biol. Chem. 279:23349-23356), meaning that M18 (K_{D}=35 pM) can effectively compete with this interaction, while 14B7 (K_{D}=4.3 nM) cannot.

Since the anti-PA M18 scAb-PEG 40 kDa lacking an Fc region is able to confer prophylactic protection against heavy challenge with inhalation anthrax spores, it is reasonable to assume that other means of inactivating anthrax toxins should also hold promise as potential therapeutics for anthrax. These include multivalent peptides that bind to PA (36), small molecule inhibitors of LF (Shoop et al. (2005) Proc. Natl. Acad. Sci. USA 102:7958-7963) and dominant negative PA mutants (Drum et al. (2002) Nature 415:396-402).

From a therapeutic development perspective, the key advantages of PEGylated antibody fragments include the ease of isolation of scFv or scAb from combinatorial libraries and the facile low-cost production by well-established techniques using E. coli. Combined with the ease of subcutaneous injection, the approach described herein represents a highly practical strategy for a large-scale prophylactic response to anthrax exposure, including antibiotic-resistant strains (Athamna et al. (2004) Antimicrob Chemother. 54:424-428). Based on the examples above, PEGylated, high-affinity antibody fragments are employable for protection or therapy against other bacterial agents where pathogenicity is intimately associated with toxin production, e.g., Shiga toxin-producing hemolytic E. coli (Tzipori, et al. (2004) Clin. Microbiol. Rev. 17:926-941), other microbial pathogens, and, finally, viral infections. Thus, PEGylated antibodies will be a practical and rapidly deployable therapeutic avenue for combating emerging infections.

Although the invention has been described with reference to the above example, it will be understood that modifications and variations are encompassed within the spirit and scope of the invention. Accordingly, the invention is limited only by the following claims.

We claim:

1. A method of protecting a subject against challenge from a pathogen or pathogen protein product thereof comprising, administering to the subject a therapeutically effective amount of an antibody fragment or functional fragment thereof, with the proviso that the fragment or functional fragment thereof does not contain an Fc region, thereby protecting the subject.

2. The method of claim 1, wherein the antibody fragment or functional fragment thereof is administered prior to or immediately following exposure of the subject to the pathogen or pathogen protein product.

3. The method of claim 1, wherein the antibody fragment or functional fragment thereof is modified to have increased circulation half-life as compared to an unmodified fragment.

4. The method of claim 1, wherein the pathogen is a bacterium.

5. The method of claim 4, wherein the pathogen is B. anthracis.

6. The method of claim 1, wherein the pathogen is B. anthracis and the pathogen protein product is anthrax protective antigen (PA) toxin.

7. The method of claim 1, wherein the antibody fragment or functional fragment thereof reduces the risk of infection to the pathogen or pathogen protein product.

8. The method of claim 1, wherein the antibody fragment or functional fragment thereof is administered subcutaneously.

9. The method of claim 1, wherein the antibody fragment or functional fragment thereof is administered intravenously.

10. The method of claim 1, wherein the antibody fragment is produced in a bacterial expression system.

11. The method of claim 1, wherein the antibody fragment or functional fragment thereof further comprises a moiety group.

12. The method of claim 11, wherein the moiety is a naturally occurring amino acid.

13. The method of claim 12, wherein the naturally occurring amino acid is cysteine.

14. The method of any of claims 11-13, wherein the moiety group is attached, conjugated or covalently linked to a non-protein polymer.

15. The method of claim 14, wherein the moiety is covalently linked to polyethylene glycol (PEG).

16. The method of claim 11, wherein the moiety is covalently linked to PEG.

17. The method of claim 16, wherein the PEG moieties are conjugated to cysteine residues on the antibody fragment.

18. The method of claim 1, wherein the antibody fragment or functional fragment thereof is an Fab, F(ab′)₂, Fv, scFv or scAb fragment.

19. The method of claim 1, wherein the antibody fragment or functional fragment thereof is the antigen binding site domain.

20. The method of claim 10, wherein the bacterial expression system is an E. coli bacterial expression system.

21. The method of claim 1, further comprising administering a therapeutic agent in addition to the antibody fragment or functional fragment thereof.

22. The method of claim 1, further comprising administering an antibiotic in combination with the antibody fragment or functional fragment thereof.

23. A method of producing an antibody fragment or functional fragment thereof that is protective when administered to a subject at risk of being infected by a pathogen or pathogen protein product thereof, comprising:
a) introducing an expression vector containing a nucleic acid encoding the antibody fragment or functional fragment protein thereof, with the proviso that the fragment or functional fragment thereof does not contain an Fc region, into the bacterial cell; and
b) culturing the bacterial cell under conditions wherein the antibody fragment or functional fragment protein thereof is expressed, thereby producing the antibody fragment that is protective when administered to a subject at risk of being infected by a pathogen or protein product thereof.

24. The method of claim 23, wherein the nucleic acid encodes a fusion protein of the antibody fragment or functional fragment thereof.

25. The method of claim 23, wherein the bacterial expression vector system is an E. coli bacterial expression system.

26. The method of claim 23, wherein the pathogen or pathogen protein product thereof is B. anthracis or anthrax protective antigen (PA).

27. The method of claim 23, further comprising testing the protective ability of the antibody fragment or functional fragment thereof in vitro or in vivo.

28. The method of claim 27, wherein the in vitro testing is on macrophages.

29. The method of claim 27, wherein the in vivo testing is in animals.

30. A method of producing an antibody fragment or functional fragment thereof that is protective against bacterial infection comprising culturing a recombinant bacterial cell containing a nucleic acid encoding an antibody fragment or functional fragment protein thereof that binds to an antigen, wherein the protein is expressed by the bacterial cell.

31. The method of claim 30, wherein the protein is recovered by a means comprising ion-exchange chromatography.

32. The method of claim 30, wherein the protein is recovered by a means comprising CM-Sephadex column chromatography.

33. The method of claim 30, wherein the protein is recovered by a means comprising DEAE-Sephadex column chromatography.

34. A purified antibody fragment or functional fragment thereof that specifically binds to anthrax protective antigen (PA) toxin, with the proviso that the antibody fragment or functional fragment thereof does not contain an Fc region, wherein the antibody fragment or functional fragment thereof is as set forth in SEQ ID NO: 1.

35. The antibody fragment or functional fragment thereof of claim 34 further comprising mutations to 121V, L46F, S56P, S76N, Q78L, and L94P of the light chain, and S30N, T57S, K64E, and T681 of the heavy chain, wherein the numbering of the amino acid is based on SEQ ID NO: 1

36. The antibody fragment or functional fragment thereof of claim 34 further comprising mutations Q38R, Q55L, S56P, T74A and Q78L of the light chain, and K62R of the heavy chain, wherein the numbering of the amino acid is based on SEQ ID NO: 1.

37. The antibody fragment or functional fragment thereof of claim 35 as set forth in SEQ ID NO: 1 and further comprising mutations S22G, L33S, Q55L, S56P, Q78L and L94P of the light chain, and S7P, K19R, S30N, T681, and M80L of the heavy chain wherein the numbering of the amino acid is based on SEQ ID NO: 1.


SEQUENCE LISTING (1 sequence)

SEQ ID NO: 1 - protein, 247 aa, Artificial sequence
  Synthetic construct
DIQMTQTTSSLSASLGDRVTISCRASQDIRNYLNWYQQKPDGTVKLLIYYTSRLQSGVPSRFSGSGSGTDYSLTISNQEQEDIGTYFCQQGNTLPWTFGGGTKLEIKRGGGGSGGGGSGGGGSGGGGSEVQLQQSGPELVKPGASVKISCKDSGYAFSSSWMNWVKQRPGQGLEWIGRIYPGDGDTNYNGKFKGKATLTADKSSSTAYMQLSSLTSVDSAVYFCARSGLLRYAMDYWGQGTSVTVSS